(19) United States
(12) Patent Application Publication
(10) Pub. No.: US 2012/0190962 A1
(43) Pub. Date: Jul. 26, 2012

(54) METHOD FOR COMPUTER-ASSISTED CONFIGURATION OF A MEDICAL IMAGING DEVICE

(76) Inventors: Karlheinz Glaser-Seidnitzer, Fuerth (DE); Werner Hauptmann, Hoehenkirchen (DE); Clemens Otte, Muenchen (DE)

(21) Appl. No.: 13/352,760

(22) Filed: Jan. 18, 2012

(30) Foreign Application Priority Data
Jan. 20, 2011 (DE) 10 2011 002 928.1

(51) Int. Cl. A61B 5/05

(52) U.S. Cl. 600/407

(57) ABSTRACT

In a method for computer-assisted configuration of a medical imaging device for examination of a patient, training data are provided that include multiple variants of protocols in the form of protocol parameter sets for operation of the imaging device. The training data also include patient-specific parameter sets with one or more features of a patient, the patient-specific parameter sets being associated with the respective protocol parameter sets. Based on the training data, relations between the protocol parameter sets and the patient-specific parameter sets are learned with a data-driven leaning method and stored as patterns in a knowledge base. In an application phase, a protocol parameter set suitable for the examination of the patient can be determined with the use of the patterns in the knowledge base, depending on features of a patient that are provided to the imaging device.

### BACKGROUND OF THE INVENTION

1. Field of the Invention

The present invention concerns a method for computer-assisted configuration of a medical imaging device, and a method to control an imaging device. Moreover, the invention concerns a medical imaging device and a computer-readable storage medium.

2. Description of the Prior Art

Different imaging devices are known from medical engineering, for example magnetic resonance tomography systems, computed tomography systems, ultrasound apparatuses and x-ray systems. For the proper operation of these complex systems, it is necessary to configure the imaging devices. For this purpose, a number of measurement parameters and other settings that are specific to the respective imaging device (but possibly also for a respective use case) must be established. The cited imaging devices require complex and comprehensive settings in order to acquire images at a desired quality or with desired properties with regard to resolution, contrast, section, size, etc.

The measurement parameters and settings that are required for the medical examination with the imaging device are typically stored as protocols. The protocols include all necessary information in order to operate the imaging device so that the medical examination can be implemented. The protocols are typically tailored to the examination to be implemented and to the properties of a specific imaging device.

In the case of a magnetic resonance tomography system, each protocol describes a sequence for image acquisition, typically with a duration of a few minutes. A sequential execution of multiple protocols or sequences is called a program. 1500 to 1800 protocols typically exist for the operation of a magnetic resonance tomography system. The number of protocols and programs is thus very comprehensive.

Each protocol contains, for example, approximately 150 parameters in the form of a protocol parameter set that defines the technical process of the image acquisition. A parameter set thus describes properties, measurement instructions or other settings for the imaging device or its software, and other work steps within the scope of the operation of the imaging device or the preparation and implementation of a medical examination. The parameters of a parameter set may be, for example, contrast agent information or other configurable settings of the imaging device, for example an echo time, acquisition time, resolution, bandwidth, turbo factor, dimensions of a field of view, slice count, slice thickness, etc.

The protocols used for examination are conventionally static in the sense that they are defined for a standard patient and are not adapted to patient-specific conditions or properties of the patient. In protocol development, the protocols are generally tested on and optimized for only a relatively small group of test subjects. In later use of the imaging device, parameter settings in the protocol possibly have to be adapted to the current situation. In particular, the weight, the age, the condition of the patient, prior illnesses, prior examinations and the like must be taken into account. For example, in the case of an overweight patient, the field of view of the imaging device must be adapted to the girth of the patient. For an examination for which the patient must hold his or her breath for a defined length of time, an appropriate protocol adaptation may be necessary. For example, infirm or very ill patients can hold their breath only for a brief period of time, which is less than the time typically required for the examination. In this case, the examination must be accelerated by adaptation of the protocol parameters, for example by a lower number of body slices of the patient being acquired.

At present, the patient-specific adaptation of protocol parameters is normally conducted manually by operators of the imaging device. This frequently leads to a non-optimal image quality in the event that the adaptation is not conducted optimally or is not conducted at all (for reasons of time, for example). An increased time is also frequently associated with such a sub-optimal examination because repeat acquisitions must be implemented due to unacceptable image quality. Moreover, the operator must have a very good understanding of the mode of operation of the imaging device in order to be able to optimally set the parameters in order to take patient-specific features into account.

Various approaches are known wherein protocols of a medical imaging system are selected or adapted automatically. In U.S. Pat. No. 7,152,785 B2, a patient-specific protocol is prepared based on patient information that is read from an identification tag of the patient.

DE 10 2008 060 719 A1 describes a method to control the acquisition operation of a magnetic resonance device in which patient-related acquisition parameters are determined. Technical activation parameters are subsequently determined automatically dependent on these patient-related acquisition parameters. The magnetic resonance device is then controlled based on these activation parameters.

In the known methods for automatic protocol selection, it has proven to be disadvantageous that appropriate rules to determine a protocol suitable for examination must be provided manually by experts and cannot be extracted automatically from known protocols.

### SUMMARY OF THE INVENTION

It is an object of the invention to simplify the configuration of an imaging device by enabling an automated and patient-specific protocol generation based on known protocols.

The method according to the invention serves for computer-assisted configuration of a medical imaging device. The imaging device can be any type of medical device with which images of the body of a patient are generated within the scope of a medical examination. In preferred embodiments, the imaging device is a magnetic resonance tomography system or a computed tomography system.

Within the scope of the method according to the invention, training data are provided that include multiple variants of protocols in the form of protocol parameter sets for operation of the imaging device, and that also include patient-specific parameter sets with one or more features of a patient, the patient-specific parameter sets being associated with the respective protocol parameter sets. A respective protocol parameter set is provided for operation of the imaging device for the examination of a patient with the feature or features of the patient-specific parameter set that is associated with the respective protocol parameter set. The respective protocol parameter set is thus suitable (and in particular optimal) for the patient according to the associated patient-specific parameter set.

Based on the training data, relations between the protocol parameter set and the patient-specific parameter sets are learned with a data-driven learning procedure and stored as a pattern in a knowledge base. In an application phase, a protocol parameter set that is suitable for the examination of the patient can be determined with the use of the pattern in the knowledge base, depending on features of a patient that are provided by the imaging device. The learned relations describe relationships between parameters of the protocol parameter set and parameters of the patient-specific parameter set. The relations may possibly also represent relationships between the parameters within the protocol parameter set or within the patient-specific parameter set.

The invention is based on the insight to extract knowledge about corresponding relations between protocol parameters and patient features from training data according to which known protocol parameter sets are associated with suitable patient-specific parameter sets, so this knowledge can subsequently be used for automatic generation of patient-specific protocols that are optimally matched to the patient to be examined. Learning methods known from the prior art—for example a statistical learning method (in particular clustering methods and/or support vector machines) and/or a learning method based on a probabilistic network (a Bayesian network, for example) and/or based on a semantic network and/or based on a neural network—can be used for learning the relationships themselves. The use of such data-driven learning methods for generation of a knowledge base (which contains knowledge with regard to the correlations between protocol parameter sets and patient-specific parameter sets) is essential to the invention.

In a preferred embodiment, the features of a patient in a patient-specific parameter set include one or more physical features of the patient, in particular weight and/or gender and/or girth or height and/or age and/or physical condition and the like. The features of the patient can also include one or more features pertaining to prior illnesses of the patient and/or previous examinations conducted on the patient.

In a further, preferred embodiment of the invention, the training data also include diagnosis-specific parameter sets with one or more diagnosis features pertaining to the diagnosis to be implemented via the imaging device, wherein a diagnosis-specific parameter set is associated with a respective protocol parameter set in the training data. A respective protocol parameter set is provided for a diagnosis based on the associated diagnosis-specific parameter set (i.e. suitably and in particular optimally). The diagnosis-specific parameter set is accounted for in the learning of the relations with the data-driven leaning method, such that a protocol suitable for the examination of the patient can be determined in the application phase with the use of the patterns of the knowledge base, dependent as well on diagnosis features provided to the imaging device. In this way, continuative medical or diagnostic questions can also be taken into account in the determination of protocols suitable for a patient examination. The questions are specified in the form of diagnosis features, for example “suspicion of perforation of the mitral leaflet”, “suspicion of cerebral hemorrhage” and the like. A very precise protocol matching is achieved with this variant of the invention, depending on a presumed illness.

In a further embodiment of the method according to the invention, rules are learned as patterns of the knowledge base with the data-driven learning method. Using these rules, a protocol suitable for the examination of the patient can then be determined depending on features of a patient that are provided to the imaging device, in particular also depending on diagnosis features provided to the imaging device.

Limitations with regard to the protocol parameter sets and/or the patient-specific parameter sets and/or the diagnosis-specific parameter sets can also advantageously be taken into account for a more precise protocol adaptation in the training with the data-driven learning method. The limitations can in particular be provided based on expert knowledge. The expert knowledge relates to the knowledge of a person who has technical understanding with regard to the mode of operation of the imaging device, and anatomical and medical understanding with regard to the acquisition of images of the human body with the use of the imaging device.

In addition to a configuration method, the invention also encompasses a method to control a medical imaging device. If the imaging device will be or is configured with the configuration method described above using the patterns of the knowledge base that is contained in the configured imaging device, a protocol suitable for the examination of the patient is determined depending on features of a patient that are provided to the imaging device. The provided patient features can be entered through a user interface of the imaging device, for example, and/or can be read out from a database.

In a preferred variant, this method is used to control an imaging device for the configuration of which diagnosis-specific parameter sets are also taken into account. In this case a protocol suitable for the examination of the patient is also determined with the aid of the pattern of the knowledge base, depending on diagnosis features provided to the imaging device.

In an embodiment of the control method according to the invention, a protocol parameter set suitable for the examination of the patient is determined by selection and/or adaptation of a provided protocol parameter set from a protocol memory of the imaging device. The adaptation of the provided protocol parameter set thereby advantageously takes place based on the establishment of parameter values of dynamic protocol parameters in the provided protocol parameter set. The provided protocol parameter set provided for adaptation can, for example, be determined from a user input or alternatively or additionally using the patterns of the knowledge base as well.

In a further embodiment of the method according to the invention, the protocol parameter set suitable for the examination of the patient is determined from case-based reasoning and/or by a locally weighted regression. These methods with which a protocol can be extracted from the knowledge base are known from the prior art. These methods are advantageously used when the patterns in the knowledge base include a number of learned protocol parameter sets for different patient-specific contexts. A protocol that is suitable for the examination of the patient can be selected or adapted by (for example) case-based reasoning from these protocol parameter sets using the nearest neighbor method.

In a further, preferred embodiment of the control method according to the invention, an online learning with the data-driven learning method is implemented during the operation of the imaging device. This takes place by updating the configuration of the imaging device by adapting a determined protocol parameter set suitable for the examination of the patient into the training data, and the data-driven learning method for updating the pattern of the knowledge base is implemented based on these training data that are supplemented with this protocol parameter set. For example, the updating can take place at predetermined time intervals or as triggered by a user.

In addition to the above methods, the invention also concerns a medical imaging device (in particular a magnetic resonance tomography system and/or a computed tomography system) that has a knowledge base that is configured with the configuration method described above, the medical imaging device also including a computer with which the method described above for control of the imaging device can be implemented.

In a preferred variant, the computer of the imaging device is designed such that it can implement the method described above for configuration of the imaging device. In this way the configuration of the imaging device can be conducted by the device itself. The online learning described above is thus enabled during the operation of the imaging device.

The invention also concerns a non-transitory, computer-readable storage medium encoded with a program code for implementation of the configuration method described above, or the control method described above, and all embodiments thereof, when the program runs on a computer.

### BRIEF DESCRIPTION OF THE DRAWINGS

The single FIGURE is a schematic representation of an embodiment of the method according to the invention for controlling an imaging device.

### DESCRIPTION OF THE PREFERRED EMBODIMENTS

The method according to the invention is explained in the following using the control of a medical imaging device in the form of a magnetic resonance tomography system, but the invention is also applicable to other medical imaging devices, for example computed tomography systems. The operation of a magnetic resonance tomography system for examination of a patient conventionally proceeds from a protocol that contains protocol parameters which indicate how the magnetic resonance tomography system is to be activated for the current examination of a patient. For the most part, static protocols that are optimized for an average patient and do not account for patient-specific conditions (for example the age, the height, the weight etc.) are presently used to operate such a tomography system. In the following Table 1, an excerpt of a conventional protocol is reproduced as an example:

TABLE 1
head \ library \ _3d \ t1_fl3d_sag_iso:

Flip angle 25 deg
Interpolation Off
Mode Inplane
Elliptical scanning On
Asymmetric echo Off
Averaging mode Long term
Elliptical filter On
Filter Prescan Normalize, Elliptical filter

Voxel size: 1.2×1.0×1.0 mm
Slices per slab 176
FoV read 260 mm
Phase resolution 85 %
Slice resolution 69 %
Bandwidth 160 Hz/Px

TR 13.0 ms

The protocol parameters of the manner in which the measurement (data acquisition) with the magnetic resonance tomograph should take place are specified and described in the EN. The specified parameters with the corresponding parameter values are known to those skilled in the art and thus need not be explained in detail herein. For example, one parameter is a “Field of View” (FOV) that indicates the spatial region of the magnetic resonance tomograph in which the measurement should take place. In order to achieve a patient-specific adaptation of the protocol, the parameter contained in said protocol presently must be modified manually by an operator of the tomography system. However, a deep understanding with regard to the mode of operation of the tomograph and the images to be acquired is required for this purpose, so such adaptations can be made only by specially trained personnel.

Within the scope of the described embodiment of the method according to the invention, an automatic adaptation of corresponding protocols is achieved based on patient-specific parameters. The adaptation takes place using a protocol that includes adaptable portions for corresponding dynamic parameters. An example of an excerpt of such a protocol which is based on the above protocol of Table 1 is shown in the following Table 2:

TABLE 2
head \ library \ _3d \ t1_fl3d_sag_iso:

Flip angle <XX> deg
<Interpolation Off>
<Mode Inplane>
Elliptical scanning <On|Off>
Asymmetric echo Off
Averaging mode <Long|Short> term
Elliptical filter <On|Off>
Filter Prescan Normalize, <Elliptical filter>

Voxel size: <XX>×1.0×1.0 mm
Slices per slab <XX>
FoV read 260 mm
Phase resolution <XX>%
Slice resolution <XX>%
Bandwidth <XX>Hz/Px

TR <XX> ms

Those parameter values that are automatically adaptable in a suitable manner are specified in angle brackets within the protocol. If values can be selected in certain value ranges, this is indicated by the characters <XX>. For other values at which only one or two selected possibilities exist, this is indicated in the table via corresponding designations, for example <Interpolation off>, <On|Off> and the like.

The dynamic establishment of the corresponding parameter values of the protocol takes place depending on patient-specific data using a knowledge-based method that models the correlations between different developments or variants of the protocol and defined, patient-specific contexts such as weight, age, condition of the patient, prior illnesses, prior examinations and the like. The knowledge-based method is thereby based on a data-driven learning method with which a corresponding knowledge base is generated, which learning method is subsequently used for adaptation of the dynamic protocol parameter, as is explained in more detail further below using the FIGURE.

The FIGURE shows an embodiment of the method according to the invention in which both a learning method for generation of the knowledge base and the protocol adaptation take place within the magnetic resonance tomograph. The knowledge base of the magnetic resonance tomograph is designated with KB in the FIGURE. To generate the knowledge base, training data in the form of previously known protocol variants PR are used, wherein patient-specific features M are associated with each protocol variant. This association thereby means that the corresponding protocol variant is very or, respectively, optimally suitable for an examination of a patient with the features M associated with the protocol variant. In the embodiment of the FIGURE, the training data are contained in the protocol memory PRD. The corresponding dynamic protocols which can be suitably adapted to features of a patient to be examined are also stored in this protocol memory.

Based on the previously known protocol variants and the features of the patient that are associated with these variants, the learning with a data-driven leaning method takes place with a computer in a magnetic resonance tomograph, with relations between the parameters of the protocol variants and the patient-specific features being determined and stored as a pattern in the knowledge base. The patterns can be represented by learned rules, Bayesian networks, semantic networks and the like, for example. The patterns are subsequently suitable for adaptation of the dynamic protocols based on features of the current patient to be examined, these features being imported or input into the magnetic resonance tomography system. Within the scope of the data-driven learning, suitably established limitations are advantageously taken into account, for example limitations in the selection of parameter value ranges depending on patient-specific features. These limitations are preferably based on expert knowledge with regard to the mode of operation of the magnetic resonance tomograph, combined with expert medical knowledge.

After the learning with the data-driven method, the knowledge base KB is thus received in the form of a memory in which the corresponding patterns are stored. Based on this knowledge base, an optimal protocol for this patient pertaining to the patient's examination can then be determined for said patient to be examined via the magnetic resonance tomograph, as is described in the following.

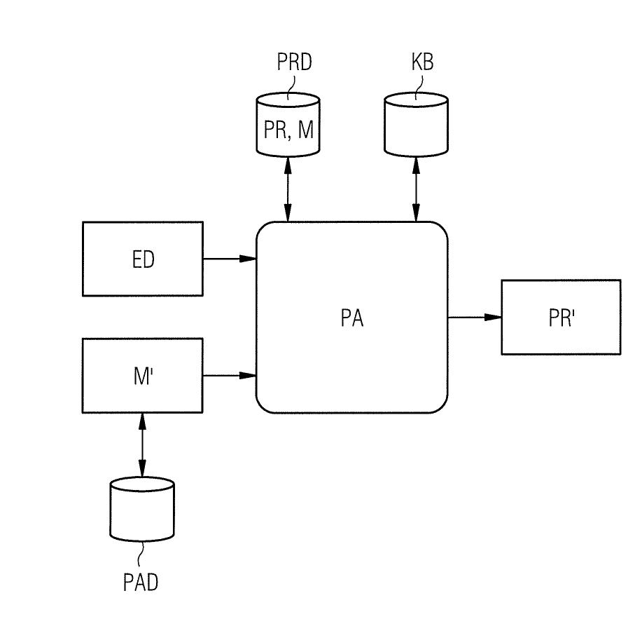

Within the scope of the examination of a patient, operating personnel of the magnetic resonance tomograph—in particular a medical technical assistant—initially makes a pre-selection of a protocol suitable for the examination of the patient. The selection is thereby made of prepared examination data, for example in paper form or possibly also in electronic form. This step is indicated with ED (=Examination Data) in FIG. 1. The selected protocol is thereby a dynamic protocol in the form of the above Table 2, meaning that some of its parameters are variable.

This protocol that is read out from the protocol memory PRD is now adapted in a suitable manner to patient-specific data within the scope of the invention, as is indicated by Step PA (PA=protocol adaptation) in FIG. 1. The patient-specific data are thereby represented by features M′.

In a preferred embodiment of the method, diagnostic or medical questions—for example a short description of the medical purpose of the examination (“suspicion of cerebral hemorrhage”, for example)—can also be input by the operator within the scope of Step ED. This information is subsequently accounted for as well in the protocol adaptation. The possibility may also exist that the operator does not make any pre-selection of a protocol at all; rather, he only inputs a corresponding medical question, whereupon a suitable protocol is selected automatically together with the patient-specific data, and its dynamic portions are adapted in Step PA. The modeling of relationships between medical questions and suitable protocols is achieved again via the patterns in the knowledge base KB, wherein corresponding, diagnosis-specific parameter sets of the training data were also taken into account in the learning of the knowledge base.

In a special embodiment of the invention, the protocol memory PRD is not considered at all within the scope of the determination of patient-specific protocols. In this case, the protocol is generated anew via the learned knowledge base based on the patient-specific data or, respectively, the examination context for each patient to be examined, without resorting to dynamic protocols. In this embodiment the protocol memory PRD includes only the protocols (with the patient-specific features associated with these protocols) used within the scope of the learning.

As was already mentioned, the protocol adaptation runs under consideration of patient-specific features M′ of the presently examined patient. These features can be read out from a patient data memory PAD. The patient data memory can thereby be an RIS information system (RIS Radiology Information System) or a HIS information system (HIS=Hospital Information System), for example. Additionally or alternatively, the possibility also exists that the operator inputs the corresponding patient-specific features via a user interface at the tomograph.

As a result of the protocol adaptation, an adapted protocol PR′ is finally obtained with which the examination of the patient is subsequently implemented. The protocol parameters obtained in the adapted protocol PR′ are thereby suitably matched to the patient to be examined, and possibly the medical question. In a preferred variant, the adapted protocol can also be adopted into the protocol memory PRD with the correspondingly associated features of the patient or, respectively, the medical questions. Within the scope of an online learning, the patterns in the knowledge base can thereby be re-learned at regular intervals or, respectively, triggered by a user, based on new training data which are supplemented with adapted protocols to be added, whereby the knowledge base becomes increasingly better adapted to the patient-specific contexts.

The method according to the invention that is described in the preceding has a number of advantages. In particular, within the scope of the examination of a patient by means of a magnetic resonance tomograph it is no longer necessary that a protocol suitable for the examination is selected purely manually by specially trained operators. Rather, a protocol can be determined automatically with protocol parameters matched to the patient, independent of the experience of the operator. A consistent image quality of the images generated via the examination is achieved in this way. Moreover, the training time for personnel is reduced to the operation of the tomograph since significantly less expertise is required for this.

Due to the automated, patient-specific determination of protocols used for examination, the time period per examination is reduced since multiple repeated image acquisition processes are avoided. Moreover, the quality of the images acquired within the scope of the examination is improved since expertise can also be suitably taken into account in the modeling of the knowledge base that is used for automatic protocol determination.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for computer-assisted configuration of a medical imaging device for examination of a patient, comprising:
providing training data to a computerized processor that comprise multiple variants of protocols as protocol parameter sets for operation of an imaging device, and patient-specific parameter sets each representing at least one feature of respectively different patients and, in said training data, said protocol parameter sets being respectively associated with said patient-specific parameter sets by means of each protocol parameter set being provided for operating the imaging device for examination of a patient having said at least one feature of the patient-specific parameter set associated therewith;
in said computerized processor, automatically using said training data to generate relations between said multiple protocol parameter sets and said patient-specific parameter sets by implementing a data-driven learning method that produces a plurality of patterns that are stored in a knowledge base; and
via the imaging device, providing a feature of a current patient to be examined with the imaging device and accessing the patterns stored in said knowledge base to determine a protocol, as a selected protocol, from among said multiple variants of protocols, that is appropriate for operating the imaging device to examine said current patient, and making the selected protocol available at an output of the processor in a form for configuring the imaging device to implement the examination of the current patient.

2. A method as claimed in claim 1 comprising, in said patient-specific parameter sets, including, in said training data, at least one patient feature selected from the group consisting of weight, gender, girth, height, age, physical condition, prior illnesses, and previous examinations.

3. A method as claimed in claim 1 comprising:
also including diagnosis-specific parameter sets in said training data respectively associated with different diagnoses that can be implemented using said imaging device, each diagnosis-specific parameter set representing at least one diagnosis feature relevant to the diagnosis associated therewith and, in said training data set, said diagnosis-specific parameter sets being respectively associated with the respective protocol parameter sets by means of a respective protocol parameter set being provided to implement the diagnosis with which the respective diagnosis-specific parameter set is associated, and wherein said computerized processor generates said patterns in said knowledge base also using said diagnosis-specific parameter sets; and
also via said imaging device, providing said computerized processor with a current diagnosis to be implemented using said examination of said current patient and determining said selected protocol using both said feature of said current patient and said current diagnosis.

4. A method as claimed in claim 3 comprising also providing data representing expert knowledge to said computerized processor, said expert knowledge comprising at least one limitation on at least one of said protocol parameter sets, said patient-specific parameter sets, and said diagnosis-specific parameter sets, and, in said computerized processor using said at least one limitation to generate said patterns that are stored in said knowledge base.

5. A method as claimed in claim 1 comprising employing, as said data-driven learning method, a method selected from the group consisting of a statistical learning method, a learning method based on a probabilistic network, a learning network based on a semantic network, and a learning network based on a neural network.

6. A method as claimed in claim 5 comprising employing a statistical learning method as said data-driven learning method and selecting said statistical learning method from the group consisting of clustering vector machines and support vector machines.

7. A method as claimed in claim 5 comprising employing a method based on a probabilistic network as said data-driven learning method, said method based on a probabilistic network being a method based on a Bayesian network.

8. A method as claimed in claim 1 comprising learning rules, as said patterns, with said data-driven learning method in said computerized processor.

9. A method as claimed in claim 1 comprising providing data to said computerized processor representing expert knowledge, said expert knowledge comprising at least one limitation on at least one of said protocol parameter sets and said patient-specific parameter sets and, in said computerized processor, using said at least one limitation to generate said patterns that are stored in said knowledge base.

10. A method for controlling a medical imaging device for examination of a patient, said medical imaging device comprising a computerized control unit, said method comprising:
providing training data to a computerized processor that comprise multiple variants of protocols as protocol parameter sets for operation of an imaging device, and patient-specific parameter sets each representing at least one feature of respectively different patients and, in said training data, said protocol parameter sets being respectively associated with said patient-specific parameter sets by means of each protocol parameter set being provided for operating the imaging device for examination of a patient having said at least one feature of the patient-specific parameter set associated therewith;
in said computerized processor, automatically using said training data to generate relations between said multiple protocol parameter sets and said patient-specific parameter sets by implementing a data-driven learning method that produces a plurality of patterns that are stored in a knowledge base;
via the imaging device, providing a feature of a current patient to be examined with the imaging device and accessing the patterns stored in said knowledge base to determine a protocol, as a selected protocol, from among said multiple variants of protocols, that is appropriate for operating the imaging device to examine said current patient, and making the selected protocol available at an output of the processor in a form for configuring the imaging device to implement the examination of the current patient; and
operating the imaging device according to the selected protocol.

11. A method as claimed in claim 10 comprising:
also including diagnosis-specific parameter sets in said training data respectively associated with different diagnoses that can be implemented using said imaging device, each diagnosis-specific parameter set representing at least one diagnosis feature relevant to the diagnosis associated therewith and, in said training data set, said diagnosis-specific parameter sets being respectively associated with the respective protocol parameter sets by means of a respective protocol parameter set being provided to implement the diagnosis with which the respective diagnosis-specific parameter set is associated, and wherein said computerized processor generates said patterns in said knowledge base also using said diagnosis-specific parameter sets; and
also via said imaging device, providing said computerized processor with a current diagnosis to be implemented using said examination of said current patient and determining said selected protocol using both said feature of said current patient and said current diagnosis.

12. A method as claimed in claim 10 comprising allowing modification of said selected protocol parameter set.

13. A method as claimed in claim 12 comprising modifying said selected protocol parameter set by establishing parameter values of dynamic protocol parameters contained in the selected protocol parameter set.

14. A method as claimed in claim 12 comprising allowing modification of said selected protocol parameter set via a user interface of said computerized control unit.

15. A method as claimed in claim 12 comprising allowing modification of the selected protocol parameter set using said patterns stored in said knowledge base.

16. A method as claimed in claim 10 comprising determining said selected protocol parameter set using a data-driven learning method selected from the group consisting of case-based reasoning and locally weighted regressions.

17. A method as claimed in claim 10 comprising updating said selected protocol parameter set by, after selecting said selected protocol parameter set, providing said selected protocol parameter set to said computerized processor as part of said training data, and re-implementing said data-driving learning method to generate an updated, selected protocol parameter set/

18. A method as claimed in claim 10 comprising embodying said computerized processor in said computerized control unit.

19. A medical imaging device for examining a patient, comprising:
an imaging apparatus configured to operate according to a protocol provided thereto to acquire medical image data from a patient;
a computerized processor provided with training data that comprise multiple variants of protocols as protocol parameter sets for operation of the imaging apparatus, and patient-specific parameter sets each representing at least one feature of respectively different patients and, in said training data, said protocol parameter sets being respectively associated with said patient-specific parameter sets by means of each protocol parameter set being provided for operating the imaging apparatus for examination of a patient having said at least one feature of the patient-specific parameter set associated therewith;
said computerized processor being configured to automatically use said training data to generate relations between said multiple protocol parameter sets and said patient-specific parameter sets by implementing a data-driven learning method that produces a plurality of patterns that are stored in a knowledge base; and
said imaging apparatus comprising a computerized control unit provided with a feature of a current patient to be examined with the imaging apparatus, said control unit being configured to access the patterns stored in said knowledge base to determine a protocol, as a selected protocol, from among said multiple variants of protocols, that is appropriate for operating the imaging apparatus to examine said current patient, and to use the selected protocol to configure the imaging apparatus to implement the examination of the current patient.

20. A medical imaging device as claimed in claim 19 wherein said computerized processor is embodied in said computerized control unit.

21. A non-transitory, computer-readable storage medium encoded with programming instructions, said storage medium being loaded into a computerized processor, and said programming instructions causing said computerized processor to configure a medical imaging device for examination of a patient, by:
receiving training data that comprise multiple variants of protocols as protocol parameter sets for operation of an imaging device, and patient-specific parameter sets each representing at least one feature of respectively different patients and, in said training data, said protocol parameter sets being respectively associated with said patient-specific parameter sets by means of each protocol parameter set being provided for operating the imaging device for examination of a patient having said at least one feature of the patient-specific parameter set associated therewith;
using said training data to generate relations between said multiple protocol parameter sets and said patient-specific parameter sets by implementing a data-driven learning method that produces a plurality of patterns that are stored in a knowledge base; and
from the imaging device, receiving a feature of a current patient to be examined with the imaging device and accessing the patterns stored in said knowledge base to determine a protocol, as a selected protocol, from among said multiple variants of protocols, that is appropriate for operating the imaging device to examine said current patient, and making the selected protocol available at an output of the processor in a form for configuring the imaging device to implement the examination of the current patient.
